(19) United States
(12) Patent Application Publication
(10) Pub. No.: US 2006/0249152 A1
(43) Pub. Date: Nov. 9, 2006

(54) Neck positioning device for mechanical ventilator

(75) Inventor: Anthony Gambone, Silver Springs, MD (US)

Correspondence Address: ROBERT D. FISH;RUTAN & TUCKER LLP, 611 ANTON BLVD 14TH FLOOR, COSTA MESA, CA, 92626-1931, US

(73) Assignee: China Resource Group, Inc.

(21) Appl. No.: 11/416,543

(22) Filed: May 2, 2006

Related U.S. Application Data
(60) Provisional application No. 60/677,528, filed on May 3, 2005.

(51) Int. Cl. G08B 5/00; F16K 31/02; A61M 16/00; A62B 7/00

(52) U.S. Cl. 128/204.18; 128/202.22; 128/202.18; 128/204.21

(57) ABSTRACT

An airway opening system has an autoinflating hyperextension cervical pillow. The pillow is placed under the next of the non-breathing victim, and is inflated in a manner that opens the airway. In preferred embodiments, the pillow is automatically inflated using air pressure from a ventilator.

This application claims priority to U.S. provisional application Ser. No. 60/677528 filed May 3, 2005.

### FIELD OF THE INVENTION

The field of the invention is breathing assistance devices.

### BACKGROUND

A common problem encountered by rescuers of unconscious, non-breathing victims, is that the victim's airway is closed. A skilled rescuer is trained to manually hyperextend the neck, and/or perform an alternative maneuver called a chin lift. Such procedures are inherently difficult to perform correctly and reliably, and the needed skill retention is generally poor. Non-skilled rescuers have little ability to perform these procedures correctly. The chief alternative in the prior art is to perform endotracheal intubation or surgical tracheotomy.

It is known to use a rolled up towel or foam for this purpose, but those solutions cannot be automatically deployed, and therefore tend to provide poor or consistent results, especially to a non-experienced user.

Thus, there is a continuing need for methods and devices that allow would-be rescuers to facilitate a patent airway in a non-breathing victim..

### SUMMARY OF THE INVENTION

The present invention provides systems and methods in which a pillow is placed under the next of the non-breathing victim, and the pillow is inflated in a manner that opens the airway. In preferred embodiments, the pillow is automatically inflated using air pressure from a ventilator.

### BRIEF DESCRIPTION OF THE DRAWING

FIG. 1 is a schematic of a breathing assist apparatus, showing a neck positioning device in a deflated configuration, and in functional positioning with respect to the head and neck of a victim.

FIG. 2 is a schematic of the apparatus and victim of claim 1, showing the neck positioning device in an inflated configuration.

### DETAILED DESCRIPTION

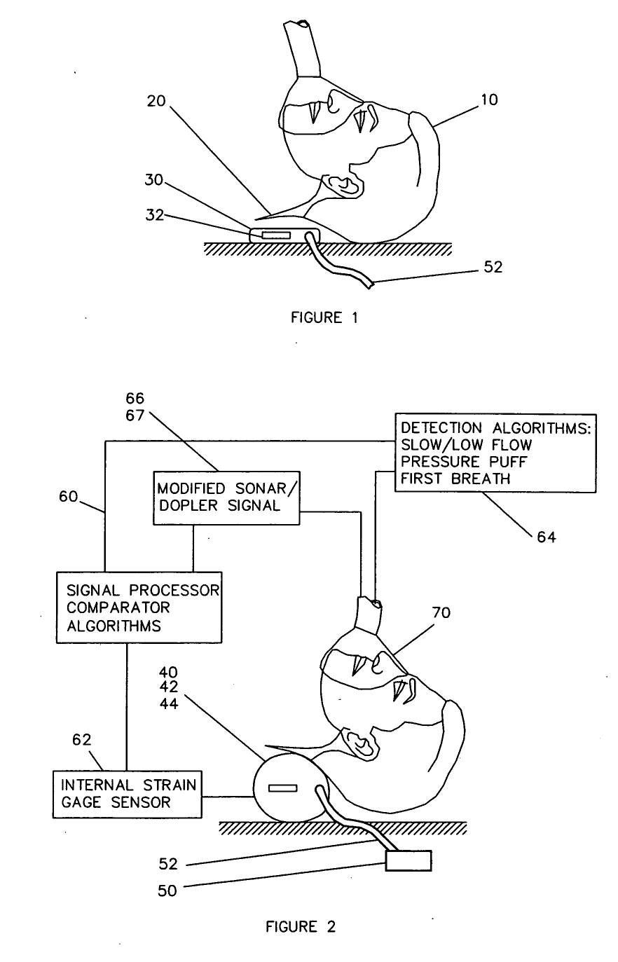

FIG. 1 generally depicts the head 10 and neck 20 of a non-breathing victim. An non-inflated pillow 30 is positioned inferior to the cervical spine.

Contemplated pillows 30 can have any suitable dimensions, including for example, a simple rectangular shape with a non-inflated length of 10-40 cm, a width of 5-30 cm, and a thickness of 0.5-2 cm. All ranges herein are inclusive of the endpoints. Other contemplated shapes include circular, oblong (oval), concave from top to bottom, double convex, convex on one side and concave on another side, and so forth. Fully inflated, the pillow 30 would likely have a greatest thickness of between 5-15 cm.

Any suitable material can be used for the pillow, including for example vinyl or other synthetic polymers. Preferred materials would be substantially puncture resistant, and would perform well despite prolonged storage in unfavorable environments.

As used herein, the status of the pillow 30 is referred to as being non-inflated regardless of its prior inflation status. Thus, the term non-inflated includes both pillows that have never been inflated, and pillows that were previously inflated and then deflated.

The pillow 30 also advantageously has a suitable warning label 32, which for example, may carry contraindications such as warning against use where there are suspected head or spinal injuries.

In FIG. 2 the pillow 40 is now inflated. Inflation is preferably accomplished using pressure from a blower that most preferably doubles as a portion of a medical ventilator 50. There is a tubing 52 that extends from the blower to the pillow 40.

Obviously, the pressure within the pillow must be enough to sufficiently hyperextend the neck. A major improvement, however, is the use of a feedback loop 60. In this particular embodiment, the feedback loop includes a first pressure sensor 62 that senses pressure of gas within the pillow 40, and a second pressure sensor 64 that senses pressure within a breathing mask 70, a sound sensor 66, and electronic circuitry 67 that alters the pressure within the pillow 40 to maintain a patent airway. Contemplated algorithms rely mostly on the sound sensor 66 to determine patency, and the pressure sensor to estimate efficiency and efficacy of breathing. The pressure sensor 62 inside the pillow is used mostly or entirely to help determine the lowest pressure at which the airway is patent.

Pillow 40 also includes an inflation/deflation adjustment valve 42. Valve 42 can be operated by the circuitry 64. A second, manually operable valve 44 can also be present.

Other types of lifters besides pillows are also contemplated. For example, one could have a ratchet mechanism (not shown) that could be manually operated, or motorized. Pillows are preferred because (a) they can be stored in a very compact manner; (b) they can be deployed rapidly; and (c) their operation can be readily and safely controlled.

It should be apparent, however, to those skilled in the art that many more modifications besides those already described are possible without departing from the inventive concepts herein. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps could be present, or utilized, or combined with other elements, components, or steps that are not expressly referenced.

What is claimed is:

1. An airway opening system comprising an autoinflating hyperextension cervical pillow.

2. The system of claim 1 further comprising a pressure sensor disposed to measure a pressure inside a lumen of the pillow.

3. The system of claim 1 wherein the pillow has a substantially rectangular shape in a non-inflated state.

4. The system of claim 1 wherein the pillow has a substantially cylindrical shape when fully inflated.

5. The system of claim 1 further comprising a face mask, and a pressurized gas source that delivers pressurized gas to both the mask and the pillow.

6. The system of claim 5 further comprising a sensor and a control circuit, the circuit using information from the sensor to at least partially control adjustment of pressure within the pillow.

7. The system of claim 5 further comprising a sensor and a control circuit, the circuit using information from the sensor to at least partially control adjustment of pressure within the pillow.
